(19) United States
(12) Patent Application Publication
(10) Pub. No.: US 2005/0250827 A1
(43) Pub. Date: Nov. 10, 2005

(54) Preparation of candesartan cilexetil in high purity

(76) Inventors: Marina Yu Etinger, Nesher (IL); Valerie Niddam-Hildesheim, Ein Vered (IL); Boris Fedotev, Haifa (IL); Ben-Zion Dolitzky, Petach Tiqva (IL)

Correspondence Address: KENYON & KENYON, ONE BROADWAY, NEW YORK, NY, 10004, US

(21) Appl. No.: 10/966,418

(22) Filed: Oct. 15, 2004

Related U.S. Application Data
(60) Provisional application No. 60/568,649, filed on May 5, 2004.

(51) Int. Cl. A61K 31/4178; C07D 43/02

(52) U.S. Cl. 514/381; 548/253

(57) ABSTRACT

The present invention is directed to the preparation of substantially pure candesartan cilexetil by the deprotection of trityl candesartan cilexetil and crystallization and/or recrystallization of candesartan cilexetil.

### RELATED APPLICATIONS

This application claims the benefit of U.S. Provisional Application No. 60/568,649, filed May 5, 2004.

### FIELD OF THE INVENTION

The present invention is directed to substantially pure candesartan cilexetil.

### BACKGROUND OF THE INVENTION

Candesartan is a potent, long-acting, selective AT₁ subtype angiotensin II receptor antagonist. Candesartan meets the requirement of high potency but it is poorly absorbed by the body when administered orally. To overcome the poor absorption, the prodrug candesartan cilexetil was developed. During absorption in the gastrointestinal tract candesartan cilexetil is rapidly and completely hydrolyzed to candesartan. The chemical name for candesartan is: 2-ethoxy-1-[[2′-(1H-tetrazol-5-yl)biphenyl-4-yl]methyl]-1H-benzimidazole-7-carboxylic acid. The chemical name for candesartan cilexetil is (±)-1-[[(cyclohexyloxy)carbonyl]oxy]ethyl-2-ethoxy-1-[[2′-(1H-tetrazol-5-yl)[1,1′-biphenyl]-4-yl]methyl]-1H-benzimidazole-7-carboxylate. Candesartan cilexetil is a white to off-white powder and is sparingly soluble in water and in methanol. Although candesartan cilexetil contains an asymmetric center in the ester portion of the molecule it is sold as the racemic mixture.

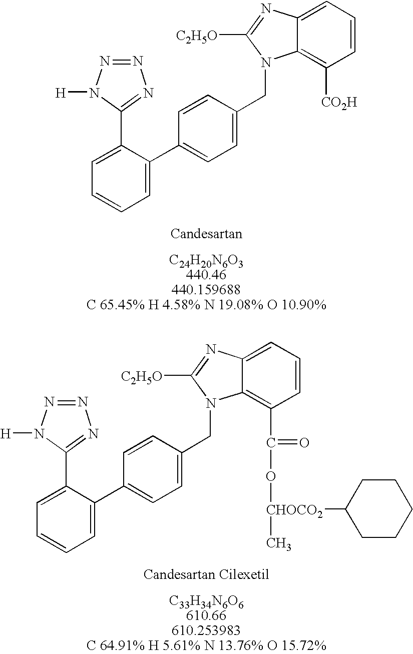

Angiotensin II is formed from angiotensin I in a reaction catalyzed by angiotensin-converting enzyme (ACE, kininase II). Angiotensin II is the principal pressor agent of the renin-angiotensin system, with effects that include vasoconstriction, stimulation of synthesis and release of aldosterone, cardiac stimulation, and renal reabsorption of sodium. Angiotensin II helps maintain constant blood pressure despite fluctuations in a person's state of hydration, sodium intake and other physiological variables. Angiotensin II also performs regulatory tasks such as inhibiting excretion of sodium by the kidneys, inhibiting norephedrin reuptake, and stimulating aldosterone biosynthesis. Candesartan blocks the vasoconstrictor and aldosterone secreting effects of angiotensin II by selectively blocking the binding of angiotensin II to the AT₁ receptor in many tissues, such as vascular smooth muscle and the adrenal gland. By inhibiting angiotensin II binding to AT₁ receptors, candesartan disrupts the vasoconstriction mediated by AT₁ receptors. Blocking vasoconstriction by angiotensin II has been found to be beneficial to patients with hypertension. The United States Food and Drug Administration has approved candesartan for the treatment of hypertension alone or in combination with other antihypertensive agents.

In U.S. Pat. No. 5,196,444, Working Example 7, 1-[[(cyclohexyloxy)carbonyl]oxy]ethyl-2-ethoxy-1-[[2′-(1H-tetrazol-5-yl)[1,1′-biphenyl]-4-yl]methyl]-1H-benzimidazole-7-carboxylate was formed by reacting 2-ethoxy-1-[[2′-(N-triphenylmethyltetrazol-5-yl)biphenyl-4-yl]methyl]benzimidazole-7-carboxylic acid in DMF with cyclohexyl 1-iodoethyl carbonate to form cilexetil trityl candesartan and deprotected with a methanolic hydrochloric acid to form candesartan cilexetil in 47% yield after column chromatography.

U.S. Pat. No. 5,578,733 discloses the deprotection of cilexetil trityl candesartan using mineral acids under substantially anhydrous conditions, water does not substantially take part in the reaction. The purification of candesartan cilexetil involved a variety of extraction steps with solvents such as ethyl acetate, ethanol, acetone, and hexane, prior to crystallizing candesartan cilexetil.

The complexity and/or high cost of the prior art procedures has created a need for a novel method of providing pure candesartan cilexetil. The present invention provides a solution to the problem presented by the prior art.

### SUMMARY OF THE INVENTION

The invention encompasses substantially pure candesartan cilexetil having less than about 0.2% by area percentage HPLC of total impurities. The invention also encompasses candesartan cilexetil having less than about 0.1% by area percentage HPLC of candesartan desethyl, preferably having less than about 0.02% by area percentage HPLC.

One embodiment of the invention encompasses processes for obtaining substantially pure candesartan cilexetil comprising providing cilexetil trityl candesartan; deprotecting the cilexetil trityl candesartan by heating to reflux cilexetil trityl candesartan in a mixture of water and methanol to obtain a residue of candesartan cilexetil; crystallizing the residue of candesartan cilexetil using methanol and toluene; and recrystallizing the crystalline candesartan cilexetil in methanol to yield a substantially pure candesartan cilexetil. Optionally, the process may further comprise drying the substantially pure candesartan cilexetil.

The process may yield substantially pure candesartan cilexetil having less than about 0.1% by area percentage HPLC of candesartan desethyl, and preferably having less than about 0.02% by area percentage HPLC of candesartan desethyl. Alternatively, the process may yield substantially pure candesartan cilexetil having less than about 0.2% by area percentage HPLC of total impurities.

Another embodiment of the invention encompasses pharmaceutical compositions comprising the substantially pure candesartan cilexetil of the invention and a pharmaceutically acceptable carrier.

### DETAILED DESCRIPTION OF THE INVENTION

The invention encompasses substantially pure candesartan cilexetil. The invention encompasses candesartan cilexetil having less than about 0.1% by area percentage HPLC of candesartan desethyl, and preferably, having less than about 0.02% by area percentage HPLC of candesartan desethyl. The invention also encompasses candesartan cilexetil having less than about 0.2% by area percentage HPLC of total impurities.

As used herein, the term “substantially pure candesartan cilexetil” refers to candesartan cilexetil having no greater than about 0.2% by weight of impurities. Preferably, the term “substantially pure candesartan cilexetil” refers to candesartan cilexetil having no greater than 0.1% weight of the CNS-desethyl. More preferably, the term “substantially pure candesartan cilexetil” refers to candesartan cilexetil having no greater than 0.02% of the CNS-desethyl. The impurity CNS-desethyl has the following structure:

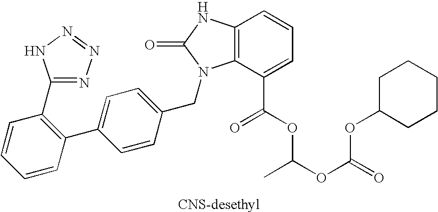

The invention also encompasses processes for obtaining substantially pure candesartan cilexetil. The process advantageously yields a substantially pure candesartan cilexetil generally free from impurities. The process comprises deprotecting trityl candesartan cilexetil by heating to reflux cilexetil trityl candesartan in a solvent mixture of water and methanol to obtain a residue of candesartan cilexetil; crystallizing the residue from a mixture of water and toluene to obtain a crystalline candesartan cilexetil; and recrystallizing the crystalline candesartan cilexetil in a second solvent system to yield a substantially pure candesartan cilexetil. As used herein, the term “crystallization” or “recrystallization” are used interchangeably regardless whether the starting material is a residue of candesartan cilexetil, a solid of candesartan cilexetil, or a crystalline form thereof.

Typically, the deprotection step comprises heating to reflux trityl candesartan cilexetil in a deprotection solvent mixture comprising water and methanol. Optionally, the deprotection solvent mixture further comprises toluene and/or formic acid. The cilexetil trityl candesartan is heated to reflux until a clear solution is obtained. Thereafter, the solvents are removed by evaporation to obtain a residue of deprotected candesartan cilexetil. The solvents may be removed at a temperature of about 30° C. to about 70° C., preferably at a temperature of about 50° C., and at a reduce pressure of about 30 mbar. As used herein, the term “residue” refers to the product obtained from the deprotection reaction. The residue candesartan cilexetil may be either a solid form or an oil form.

During the recrystallization step, candesartan cilexetil residue is dissolved in a minimal amount of methanol and toluene; thereafter, the solution is cooled slowly until a crystalline candesartan cilexetil precipitate appears. Crystallization may be induced by seeding, etching, cooling, or other techniques commonly known to one of ordinary skill in the art. Optionally, during the crystallization or recrystallization step, the solution may be stirred. Thereafter, the crystalline candesartan cilexetil obtained during the first crystallization is allowed to dry. The drying step may be performed by heating the crystalline candesartan cilexetil, optionally under reduced pressure, until a constant weight is obtained. Typically, drying is performed at a temperature of about 45° C. to about 65° C., and preferably at a temperature of about 50° C. to about 60° C. When present, the reduce pressure includes, but is not limited to, about 30 mbar.

Generally, the solvent mixture for crystallization comprises methanol and toluene present in a ratio of about 20% methanol to 80% toluene by weight; preferably, the ratio of methanol to toluene is about 10% methanol to 90% toluene by weight of the solvent mixture. More preferably, the weight ratio of methanol to toluene is about 5% methanol to 95% toluene by weight.

The recrystallizing of crystalline candesartan cilexetil comprises dissolving the crystalline candesartan cilexetil in methanol and recrystallizing to obtain a substantially pure candesartan cilexetil. Optionally, during the recrystallization, the solution may be stirred.

Optionally, the process may further comprise a drying step wherein after the second recrystallization, the substantially pure candesartan cilexetil is dried at a suitable temperature and for a suitable time to obtain a substantially pure dry candesartan cilexetil of a constant weight. Generally, the drying temperature should be sufficient to remove undesired solvents until the weight of the crystalline candesartan cilexetil does not fluctuate. For example, the drying temperature may be about 50° C. to 65° C., and preferably, the drying temperature is about 50° C. Optionally, the drying step may be performed at a reduced pressure including, but not limited to, about 8 mbar.

The crystalline candesartan cilexetil (CNS) obtained using the process of the invention was compared to commercially available candesartan cilexetil obtain as ATACAND® from AstraZeneca LP. (Wilmington, Del.). The candesartan cilexetil prepared using the processes of the invention, i.e., Samples 1 and 2 (corresponding to examples 1 and 2, respectively) had less impurities as compared to the commercially available candesartan cilexetil (Sample 4). The results are summarized in Table 1. The impurities are measured by the relative retention time (RRT) of each impurity using HPLC analysis. As illustrated in Table 1, the purified samples of the invention contain less CNS-desethyl (0.02%) than the commercially available sample (0.13%). In fact, the commercially available candesartan cilexetil has six (6) times more of the impurity and less candesartan cilexetil.

TABLE 1
Comparative Data of Examples 1-3
and Commercially available CNS

| Example | CNS-desethyl (RRT 0.60) | CNS (% wt) |
|---|---|---|
| 1 | 0.02 | 99.82 |
| 2 | 0.02 | 99.83 |
| 3 (Comparative) | 0.24 | 98.01 |
| Commercial tablet | 0.13 | 99.62 |

Having described the invention with reference to certain preferred embodiments, other embodiments will become apparent to one skilled in the art from consideration of the specification. The invention is further defined by reference to the following examples describing in detail the preparation of the composition and methods of deprotection of the invention. It will be apparent to those skilled in the art that many modifications, both to materials and methods, may be practiced without departing from the scope of the invention.

### EXAMPLES

Each sample was analyzed using an HPLC to determine the content of impurities and candesartan cilexetil. The HPLC settings used included a column and packing of Luna C18 4.6×250 mm, 5 μm. The eluent consisted of a mixture of two eluents in a 70% to 30%, of a first eluent to a second eluent, respectively. The first eluent is 0.1% trifluoroacetic acid in acetonitrile and the second eluent is 0.1% trifluoroacetic acid in water. The detector was set for 215 nm, and the flow was regulated to 1 ml/min.

### Example 1

### Synthesis of Substantially Pure Candesartan Cilexetil

A suspension of cilexetil trityl candesartan (50.0 g, 58.62 mmol), water (2.64 g, 2.5 eq), and methanol (500 ml, 10 eq. by volume) was refluxed for about 16.5 h to obtain a clear solution. The solvents were removed by evaporation at 30 mbar and 40° C. to obtain a solid residue (51.7 g). The residue was dissolved at 60° C. in a mixture of toluene/methanol (95:5 w/w, 125 g), cooled to 20-23° C. and stirred for about 15 h. A precipitate appeared and was collected by filtration, washed with a cold (4° C.) mixture of toluene/methanol (95:5 w/w, 25 g), and dried for 2 h at 50° C. and 30 mbar to give a crude solid candesartan cilexetil (32.41 g, 90.5%).

The crude candesartan cilexetil (32.0 g) was dissolved at 50° C. in methanol (160 g, 5 w), the solution was filtered and stirred at 20-25° C. for about 15 h. The solids were filtered off, washed with methanol (32 g) to give a wet product (25 g), which was dried for about 1 h at 50° C. to give 21.1 g of white solid (66%). The solid was identified as candesartan cilexetil in 99.82% purity as determined by HPLC.

### Example 2

### Synthesis of Substantially Pure Candesartan Cilexetil

A solution of cilexetil trityl candesartan (30.0 g, 0.035 mol) and formic acid (1.6 g, 0.035 ml) in toluene (180 ml), and methanol (180 ml) was refluxed. After about 10 h, the solvents were evaporated at 60° C. and 30 mbar. The resulting oily residue was dissolved in a mixture of toluene/methanol 90:10 (w/w, 73 g), and the mixture was cooled at 4° C. to 7° C. for about 20 h. The solids were collected by filtration, washed with a mixture of toluene/methanol 90:10 (w/w, 15 g), and dried at 60° C. and 30 mbar to a constant weight to give candesartan cilexetil as a white solid (16.88 g, 78.6%).

The crude candesartan cilexetil (5.0 g) was dissolved at 19-22° C. in methanol (25 g) to obtain a clear solution. A precipitate began to form in about 10 min. The mixture was stirred at 19-22° C. for about 60 h. The solids were collected by filtration, washed with a cold methanol (2.5 g), and dried at 50° C. and 8 mbar to obtain a constant weight of candesartan cilexetil as a white solid (4.20 g, 84.0%) which was 99.83% pure by HPLC.

### Example 3

### Reproduction of U.S. Pat. No. 5,578,733

Cilexetil trityl candesartan (4.0 g) was dissolved at 20-25° C. in dichloromethane (DCM, 15.4 g, 11.6 ml), methanol (7.3 g, 9.2 ml) was added and the solution was cooled to 5° C. Then, a solution of HCl (gas, 0.21 g) in methanol (1.9 g, 2.4 ml) was added dropwise over a period of 15 min. The mixture was stirred at 5° C. for about 3.5 h (TLC-control), and ethyl acetate (7.6 ml) and water (7.6 ml) were added. The pH of the mixture was adjusted to pH 6.5 with a saturated aq. solution of sodium bicarbonate, followed by addition of ethyl acetate (4 ml) and 20% aq. sodium chloride (4 ml). The aqueous solution was separated and extracted with ethyl acetate (8 ml). The ethyl acetate layers were combined and redistributed in 20% aq. sodium chloride (4 ml) and ethyl acetate (4 ml). The organic layer was separated and concentrated to obtain a residue (4.4 g). Ethanol (20 ml) was added to the residue and the residue was evaporated to dryness to obtain a semi-solid residue. Ethanol (10 ml) was added to the semi-solid residue and the mixture was stirred to obtain a fine suspension. Acetone (8 ml) was added to the suspension, the suspension was stirred at 20-25° C. for about 3 h, and hexane (36 ml) was added. The solution was stirred at 20-25° C. for about 1 h and then at 4-8° C. for about 2 h. The solids were collected by filtration, washed with a cold mixture of acetone/hexane (1:9 v/v, 10 ml), and dried at 30° C. and 10 mbar to obtain a constant weight of candesartan cilexetil as a white solid (0.94 g, 32%), which was 98.01% pure by HPLC (CNS-desethyl impurity 0.24%).

1. A substantially pure candesartan cilexetil having less than about 0.1% by area percentage HPLC of candesartan desethyl.

2. The substantially pure candesartan cilexetil according to claim 1 having less than about 0.02% by area percentage HPLC of candesartan desethyl.

3. A substantially pure candesartan cilexetil has less than about 0.2% by area percentage HPLC of total impurities.

4. A process for obtaining substantially pure candesartan cilexetil comprising:
providing cilexetil trityl candesartan;
deprotecting the cilexetil trityl candesartan by heating to reflux cilexetil trityl candesartan in a mixture of water and methanol to obtain a residue of candesartan cilexetil;
crystallizing the residue of candesartan cilexetil using methanol and toluene; and
recrystallizing the crystalline candesartan cilexetil in methanol to yield a substantially pure candesartan cilexetil.

5. The process according to claim 4 further comprising drying the substantially pure candesartan cilexetil.

6. The process according to claim 4, wherein the substantially pure candesartan cilexetil has less than about 0.1% by area percentage HPLC of candesartan desethyl.

7. The process according to claim 4, wherein the substantially pure candesartan cilexetil has less than about 0.02% by area percentage HPLC of candesartan desethyl.

8. The process according to claim 4, wherein the substantially pure candesartan cilexetil has less than about 0.2% by area percentage HPLC of total impurities.

9. Candesartan cilexetil prepared by the process of any one of claims 4 to 8.

10. A pharmaceutical composition comprising the candesartan cilexetil of any one of claims 1 to 3 and a pharmaceutically acceptable carrier.
